(19) United States
(12) Patent Application Publication
(10) Pub. No.: US 2025/0302405 A1
(43) Pub. Date: Oct. 2, 2025

(54) MEDICAL IMAGE GENERATION METHOD, ELECTRONIC DEVICE AND STORAGE MEDIUM

(71) Applicant: Shanghai United Imaging Healthcare Co., Ltd., Shanghai (CN)

(72) Inventor: Xiaochun Xu, Shanghai (CN)

(21) Appl. No.: 19/097,899

(22) Filed: Apr. 2, 2025

(30) Foreign Application Priority Data
Apr. 2, 2024 (CN) 202410398624.7

(51) Int. Cl. A61B 6/03; A61B 6/00; G16H 30/40

(52) U.S. Cl. CPC A61B 6/037; A61B 6/488; A61B 6/5258; G16H 30/40

(57) ABSTRACT

A medical image generation method is provided. A PET dynamic image of a target part of a scanned object is obtained, a quality analysis is performed on the PET dynamic image to determine a first single frame image that meets quality enhancement requirements, the first single frame image corresponding to an initial single frame duration. A second set of scanning data corresponding to a first single frame duration of the PET dynamic image are obtained when a count of coincidence events corresponding to the first single frame image is less than a count threshold. The first single frame duration is obtained by extending the initial single frame duration. A target single frame image of the target part is constructed based on the second set of scanning data, and the count of coincidence events corresponding to the second set of scanning data is greater than or equal to the count threshold.

### CROSS-REFERENCE TO RELATED APPLICATIONS

The present application claims priority to Chinese patent application No. 202410398624.7, filed on Apr. 2, 2024, and entitled “MEDICAL IMAGE GENERATION METHOD, ELECTRONIC DEVICE AND STORAGE MEDIUM”, the entire content of which is incorporated herein by reference.

### TECHNICAL FIELD

The present disclosure relates to the field of medical imaging technologies, and in particular, to a medical image generation method, electronic device and storage medium.

### BACKGROUND

According to literature statistics, 80% of positron emission tomography (PET)/computed tomography (CT) images have quality problems. The quality of PET images has a direct impact on the stability of parameter map output. However, the current image quality control is only done manually to find problems with the reconstructed image. When quality problems are found in the reconstructed image, the image needs to be reconstructed based on the acquired data. For a dynamic image that takes up to one hour to acquire, the actual output time will be even longer. If there is a problem with the image, a lot of time will be needed to rebuild the image, which affects the doctor's work efficiency.

### SUMMARY

The present disclosure relates to a medical image generation method, electronic device and storage medium.

In a first aspect, a medical image generation method is provided in the present disclosure. The method includes:
obtaining a PET dynamic image of a target part of a scanned object;
performing quality analysis on the PET dynamic image to determine a first single frame image that meets quality enhancement requirements, the first single frame image corresponding to an initial single frame duration;
obtaining a second set of scanning data corresponding to a first single frame duration of the PET dynamic image when a count of coincidence events corresponding to the first single frame image is less than a count threshold, the first single frame duration being obtained by extending the initial single frame duration; and
constructing a target single frame image of the target part based on the second set of scanning data, a count of coincidence events corresponding to the second set of scanning data being greater than or equal to the count threshold.

In some embodiments, constructing a target single frame image of the target part based on the second set of scanning data includes:
determining an intermediate single frame image of the target part based on the second set of scanning data;
obtaining a third set of scanning data corresponding to a second single frame duration when a SNR of the intermediate single frame image is less than a SNR threshold, the second single frame duration being obtained by extending the first single frame duration; and
constructing the target single frame image based on the third set of scanning data, a SNR corresponding to the target single frame image being greater than the SNR threshold.

In some embodiments, the medical image generation method includes:
determining the count threshold based on body parameters of the scanned object.

In some embodiments, determining the count threshold based on the body parameters of the scanned further includes:
obtaining a pre-stored correspondence and the body parameters, the correspondence including a correspondence between the body parameters and the count threshold; and
determining the count threshold based on the body parameters and the correspondence.

In some embodiments, the medical image generation method includes:
determining an SUV value of the target single frame image;
normalizing the SUV value to obtain a normalized SUV value of the target single frame image; and
determining a lesion condition of the target part based on the normalized SUV value.

In some embodiments, determining the lesion condition of the target part based on the normalized SUV includes:
obtaining a normalized SUV value corresponding to a reference lesion condition;
comparing the normalized SUV value of the target single frame image with the normalized SUV value corresponding to the reference lesion condition to obtain a comparison result; and
determining the lesion condition of the target part based on the comparison result.

In some embodiments, the medical image generation method further includes:
obtaining a corresponding scanning protocol corresponding to the target part, and locating the target part, in response to obtaining a scanning instruction for the target part; and
performing a PET dynamic scan on the target part according to the scanning protocol.

In some embodiments, the medical image generation method further includes:
outputting prompt information for prompting a user to determine whether to adjust a scanning duration; and
adjusting the scanning duration based on the second single frame duration when trigger information for adjusting the scanning duration is obtained.

In some embodiments, obtaining a PET dynamic image of a target part of a scanned object includes:
determining the target part of the scanned object;
performing a PET dynamic scan on the target part to obtain a PET image sequence of the target part; and
processing the PET image sequence through an image reconstruction algorithm to obtain the PET dynamic image.

In some embodiments, the quality enhancement requirements include one or more of the following: an SUV value, a SNR, a coefficient of variation, or a noise equivalent count being less than a set value, or artifacts exceeding a threshold range.

In a second aspect, a medical image generation apparatus is provided in the present disclosure. The apparatus includes:
a first obtaining module configured to obtain a PET dynamic image of a target part of a scanned object;
an analysis module configured to determine a first single frame image that meets quality enhancement requirements, the first single frame image corresponding to an initial single frame duration;
a third obtaining module configured to obtain a second set of scanning data corresponding to a first single frame duration of the PET dynamic image when a count of coincidence events corresponding to the first single frame image is less than a count threshold, the first single frame duration being obtained by extending the initial single frame duration; and
a construction module configured to construct a target single frame image of the target part based on the second set of scanning data, a count of coincidence events corresponding to the second set of scanning data being greater than or equal to the count threshold.

In a third aspect, an electronic device is provided in the embodiments of the present disclosure. The electronic device includes a memory and a processor, the memory storing a computer program. The processor, when executing the computer program, performs a medical image generation method which includes:
obtaining a PET dynamic image of a target part of a scanned object;
performing quality analysis on the PET dynamic image to determine a first single frame image that meets quality enhancement requirements, the first single frame image corresponding to an initial single frame duration;
obtaining a second set of scanning data corresponding to a first single frame duration of the PET dynamic image when a count of coincidence events corresponding to the first single frame image is less than a count threshold, the first single frame duration being obtained by extending the initial single frame duration; and
constructing a target single frame image of the target part based on the second set of scanning data, a count of coincidence events corresponding to the second set of scanning data being greater than or equal to the count threshold.

In a fourth aspect, a computer-readable storage medium having a computer program stored thereon is provided in the embodiments of the present disclosure. The computer program, when executed by the processor, causes the processor to perform a medical image generation method which includes:
obtaining a PET dynamic image of a target part of a scanned object;
performing quality analysis on the PET dynamic image to determine a first single frame image that meets quality enhancement requirements, the first single frame image corresponding to an initial single frame duration;
obtaining a second set of scanning data corresponding to a first single frame duration of the PET dynamic image when a count of coincidence events corresponding to the first single frame image is less than a count threshold, the first single frame duration being obtained by extending the initial single frame duration; and
constructing a target single frame image of the target part based on the second set of scanning data, a count of coincidence events corresponding to the second set of scanning data being greater than or equal to the count threshold.

In a fifth aspect, a computer program product is provided in the embodiments of the present disclosure. The computer program product, when running on an electronic device, causes the electronic device to perform a medical image generation method which includes:
obtaining a PET dynamic image of a target part of a scanned object;
performing quality analysis on the PET dynamic image to determine a first single frame image that meets quality enhancement requirements, the first single frame image corresponding to an initial single frame duration;
obtaining a second set of scanning data corresponding to a first single frame duration of the PET dynamic image when a count of coincidence events corresponding to the first single frame image is less than a count threshold, the first single frame duration being obtained by extending the initial single frame duration; and
constructing a target single frame image of the target part based on the second set of scanning data, a count of coincidence events corresponding to the second set of scanning data being greater than or equal to the count threshold.

### BRIEF DESCRIPTION OF THE DRAWINGS

In order to describe the technical solutions in the embodiments of the present disclosure or the conventional technology more clearly, the following will briefly introduce the accompanying drawings required for describing the embodiments or the conventional technology. Apparently, the accompanying drawings in the following description are merely embodiments of the present disclosure, and for a person of ordinary skill in the art, other drawings can be obtained based on the disclosed drawings without creative efforts.

FIG. 1 is a flowchart of a medical image generation method provided in the embodiments of the present disclosure.

FIG. 2 is a flowchart of steps of constructing a target single frame image of a target part based on a second set of scanning data provided in an embodiment of the present disclosure.

FIG. 3 is a flowchart of steps of determining a lesion condition of a target part provided in an embodiment of the present disclosure.

FIG. 4 is a flowchart of steps of obtaining a comparison result provided in an embodiment of the present disclosure.

FIG. 5 is a flowchart of steps of performing a PET dynamic scan on a target part provided in an embodiment of the present disclosure.

FIG. 6 is a flowchart of steps of adjusting a scanning duration provided in an embodiment of the present disclosure.

FIG. 7 is a schematic diagram of a configuration of a medical image generation apparatus provided in an embodiment of the present disclosure.

FIG. 8 is a schematic diagram of a configuration of an electronic device provided in an embodiment of the present disclosure.

In the accompanying drawings, the same reference numerals are used for the same components, and the accompanying drawings are not drawn to scale.

### DETAILED DESCRIPTION OF THE EMBODIMENTS

The technical solutions in the embodiments of the present disclosure will be clearly and completely described with reference to the accompanying drawings. Apparently, the described embodiments are only some but not all of the embodiments of the present disclosure. Based on the embodiments of the present disclosure, all other embodiments obtained by a person of ordinary skill in the art without creative efforts shall fall within the protection scope of the present disclosure. In the following description, the term ‘some embodiments’ refers to a subset of all possible embodiments. It is understood that ‘some embodiments’ may constitute the same subset or different subsets of all possible embodiments, and they can be combined with each other without conflicts.

If similar descriptions of “first\second\third” appear in the present disclosure, the following instructions are added. In the following description, the terms “first\second\third” involved are merely used to distinguish similar objects and do not represent a specific ordering among the objects. It can be understood that “first\second\third” can be interchanged in a specific order or sequence where permitted, so that the embodiments of the present disclosure described herein can be implemented in an order other than that illustrated or described herein.

Unless otherwise defined, all technical and scientific terms used herein have the same meaning as those commonly understood by those skilled in the art to which the present disclosure belongs. The terms used herein in the specification of the present disclosure are only for the purpose of describing specific embodiments and are not intended to limit the present disclosure.

Before introducing the embodiments of the present disclosure, a brief introduction to the problems in the related art is described. PET dynamic scanning is an imaging technology that continuously collects data to observe the changes of biological processes in the body over time. The frame time of extraordinary dynamic image scanning is fixed, generally 2 seconds per frame in the first minute, then 10 seconds per frame for 1-3 minutes, and 30 seconds per frame for 3-6 minutes. These short frame times result in poor image quality. When quality problems are found in a reconstructed image, the image needs to be reconstructed based on acquired data. For a dynamic image with an acquisition time of up to one hour, the actual image output time will be longer. If there is a problem with the image, a lot of time will be required to reconstruct the image, reducing the doctor's work efficiency.

Based on the problems existing in the related art, the embodiment of the present disclosure provides a medical image generation method, which can be applied to electronic devices such as mobile phones, tablet computers, wearable devices, vehicle-mounted devices, augmented reality (AR)/virtual reality (VR) devices, laptops, ultra-mobile personal computers (UMPCs), netbooks, personal digital assistants (PDAs), or scanning devices. The embodiment of the present disclosure does not impose any limitations on the specific types of electronic devices.

In the embodiments of the present disclosure, the scanning device may be a single-modality device, such as a positron emission tomography (PET) scanning device. In some embodiments, the scanning device may also be a multi-modality device, such as a PET/CT device, a PET/MR device, etc.

The medical image generation method provided in the embodiments of the present disclosure can be implemented by calling program instructions by a processor of an electronic device. The program instructions can be stored in a computer storage medium.

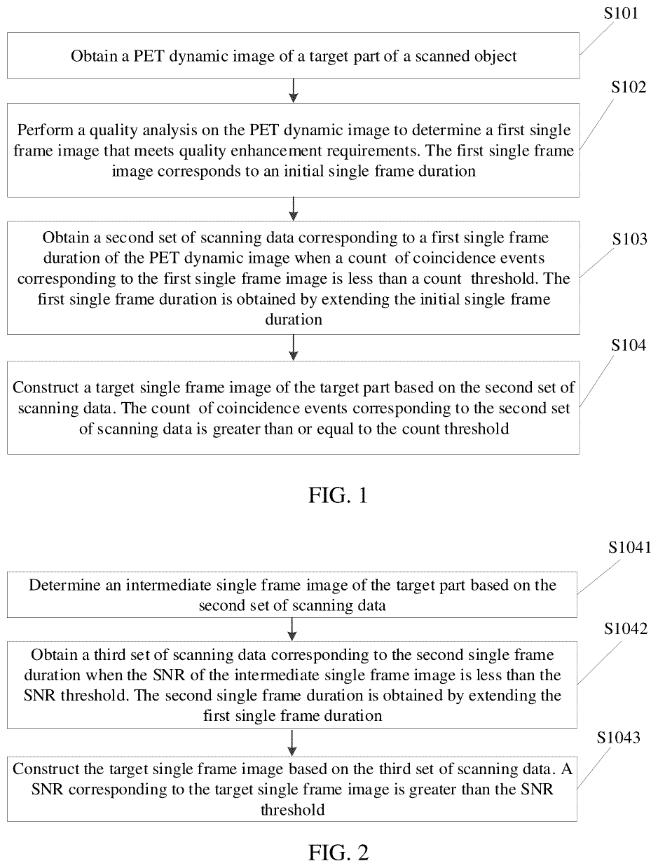

A medical image generation method is provided in the present disclosure. FIG. 1 is a flowchart of a medical image generation method provided in the embodiments of the present disclosure. As shown in FIG. 1, the method includes the following steps S101 to S104.

In step S101, a PET dynamic image of a target part of a scanned object is obtained.

In the embodiments of the present disclosure, the scanned object may be a patient, and the target part may be any part of the head, chest, abdomen, etc.

In the embodiments of the present disclosure, the PET dynamic image can be a three-dimensional image. The PET dynamic image can be obtained by determining the target part of the scanned object by, for example, medical imaging, and scanning the target part of the scanned object by the PET scanning device, which usually requires injection of radioactive tracers. Under set scanning conditions, a PET scan is performed to obtain a PET image sequence of the target part, and the data obtained by the PET scan, such as the PET image sequence, is converted into a PET dynamic image through an image reconstruction algorithm.

In some embodiments, the target part of the scanned object is determined by medical imaging, including obtaining anatomical images by high-resolution CT (Computed Tomography) or MRI (Magnetic Resonance Imaging) to determine the target part of the scanned object.

In some embodiments, a PET dynamic image of the target part of the scanned object can be obtained through a network.

In some embodiments, a PET dynamic image of the target part can be obtained through a storage device.

In step S102, a quality analysis is performed on the PET dynamic image to determine a first single frame image that meets quality enhancement requirements. The first single frame image corresponds to an initial single frame duration.

In the embodiments of the present disclosure, performing the quality analysis includes one or more of the following: an SUV (Standard Uptake Value) analysis, a signal-to-noise ratio (SNR) analysis, a coefficient of variation analysis, and a noise equivalent count rate analysis, or an artifacts analysis.

In the embodiments of the present disclosure, the PET dynamic image can be input into a neural network model to perform quality analysis and obtain a quality analysis result. In some examples, the neural network model can include one or more of the following: an SUV analysis model, a SNR analysis model, a coefficient of variation analysis model, a noise equivalent count rate analysis model, or an artifact analysis model.

In the embodiments of the present disclosure, a sample PET dynamic image can be obtained, sample PET dynamic image data can be preprocessed and feature extracted to meet input requirements of the neural network model, and the sample PET dynamic image can be marked. A neural network model suitable for processing PET dynamic images is designed and constructed, which may include structures such as convolutional neural networks (CNNs). The constructed neural network model is trained using a labeled PET dynamic image dataset to learn quality characteristics of a PET image. The trained neural network model is evaluated through a validation set or a test set to check its performance on a quality analysis task. The trained neural network model is applied to new PET dynamic image data for quality analysis, and a quality analysis result is obtained.

In the embodiments of the present disclosure, the quality analysis result obtained by performing the quality analysis on the PET dynamic image may include: whether one or more of the following: an SUV value, a SNR, a coefficient of variation, a noise equivalent count rate, etc. is less than a set value, or whether artifacts exceed a threshold range.

In some embodiments, the first single frame image corresponding to an initial single frame duration of the PET dynamic image is obtained based on the quality analysis result. The quality analysis result may include that a first single frame image that meets the quality enhancement requirements is obtained or not obtained. If the first single frame image that meets the quality enhancement requirements is not obtained, it indicates that the PET dynamic image meets quality requirements, and the first single frame image can be determined as the target single frame image.

In the embodiments of the present disclosure, on a condition that the quality analysis result indicates that one or more of quality analysis results of the first single frame image is less than the set value, or the artifacts exceed the threshold range, it is considered that the first single frame image meets the quality enhancement requirements. Specifically, the quality enhancement requirements may include one or more of the following: an SUV value, a SNR, a coefficient of variation, or a noise equivalent count rate being less than the set value, or the artifacts exceeding the threshold range.

In the embodiments of the present disclosure, a preset acquisition duration can be configured. When performing a scan and reconstruction of the PET dynamic image, the doctor can set the preset acquisition duration. In the embodiments of the present disclosure, the working principle of PET is that a drug containing a radioactive nuclide is injected into an object under a scan, the radioactive nuclide decays to produce a positron, the positron annihilates with the surrounding negative electrons to produce a pair of back-to-back gamma photons, the gamma photons pass through the object under a scan and reach a PET detector to be received and recorded, the detector receives coincidence events, and reconstructs the image based on the coincidence events to obtain a distribution map of the nuclide emitting the positron.

In the embodiments of the present disclosure, the user will receive an injection of a radioactive tracer, which will emit positrons in the body. After the radioactive tracer is fully distributed to the target part, a scan is performed. During the scan, the user is positioned in a scanning device. The scanning device is configured to detect and record coincidence events emitted by the tracer, which are captured by a highly sensitive camera, thereby obtaining the first set of scanning data corresponding to the preset acquisition duration.

In some embodiments, the preset acquisition duration can be n times the initial single frame duration, and the initial single frame duration can be preset. For example, the initial single frame duration can be set to 2 seconds.

In the embodiments of the present disclosure, a count rate is a number of coincidence events detected per unit time. The count of coincidence events corresponding to the first single frame image can be calculated based on the first set of scanning data. The scanning device records the distribution of the radioactive tracer in the patient's body, and then calculates the number of coincidence events detected per unit time based on the half-life of the radioactive tracer and the sensitivity of the detector to obtain the count rate.

In step S103, a second set of scanning data corresponding to a first single frame duration of the PET dynamic image are obtained when a count of coincidence events corresponding to the first single frame image is less than a count threshold. The first single frame duration is obtained by extending the initial single frame duration.

In the embodiments of the present disclosure, since different body parameters may correspond to different physiological characteristics, the count threshold may also vary from person to person, and the count threshold may be determined based on the body parameters of the scanned object.

In the embodiments of the present disclosure, the second set of scanning data can be obtained on a condition that a count rate of coincidence events corresponding to the first single frame image is less than a count rate threshold.

In the embodiments of the present disclosure, the count threshold or the count rate threshold can be set by user based on the body parameters of the scanned object. The body parameters include data related to the physiological characteristics, health status or body composition of the scanned object, such as age, height, gender, weight, blood pressure, heart rate, etc.

In the embodiments of the present disclosure, by adjusting the single frame duration, set of scanning data corresponding to a plurality of single frame durations can be obtained, thereby obtaining multiple frame images.

In some embodiments, before step S103, the method further includes determining the count threshold or the count rate threshold based on the body parameters of the scanned object.

In some embodiments, determining the count threshold based on the body parameters of the scanned includes: obtaining a pre-stored correspondence and the body parameters, and determining the count threshold based on the body parameters and the correspondence. The correspondence includes a correspondence between the body parameters and the count threshold.

In some embodiments, determining the count rate threshold based on the body parameters of the scanned includes: obtaining a pre-stored correspondence and the body parameters, and determining the count rate threshold based on the body parameters and the correspondence. The correspondence includes a correspondence between the body parameters and the count rate threshold.

In the embodiments of the present disclosure, the body parameters may include age, gender, and weight, etc.

In the embodiments of the present disclosure, medical professionals can establish a correspondence between age, gender, weight and the count threshold, or a correspondence between age, gender, weight and the count rate threshold, based on clinical experience and relevant research, and then store it in an electronic device. After obtaining age, gender and weight, the correspondence can be called to determine the count threshold or the count rate threshold.

In the embodiments of the present disclosure, in a PET scan, on a condition that the count rate of coincidence events corresponding to the first single frame image is less than a preset count rate threshold, it is usually considered to adjust the initial single frame duration to obtain more data. A low count rate may indicate that the distribution of the tracer is not ideal or the SNR of the scan is unsatisfying, which may affect the image quality and the recognition of abnormal conditions. Therefore, it is necessary to extend the initial single frame duration to obtain the first single frame duration.

In the embodiments of the present disclosure, the extension of the initial single frame duration can be made based on a time step. The time step can be set to be, for example, 0.5 seconds.

In the embodiments of the present disclosure, the extended duration can vary based on the type of the PET scanning device and the specific process of the medical institution.

In the embodiments of the present disclosure, the first single frame time can be set as 2.5 seconds.

In the embodiments of the present disclosure, since the first single frame duration is longer than the initial single frame duration, the amount of the second set of scanning data may be more than that of the first single frame image, and more counts of coincidence events may be obtained.

In step S104, a target single frame image of the target part is constructed based on the second set of scanning data. The count of coincidence events corresponding to the second set of scanning data is greater than or equal to the count threshold.

In the embodiments of the present disclosure, the count of coincidence events corresponding to the second set of scanning data can be determined based on the second set of scanning data. On a condition that the count of coincidence events corresponding to the second set of scanning data is less than the count threshold, the initial single frame duration is further extended. The second set of scanning data corresponding to the extended initial single frame duration are obtained until the obtained count of coincidence events corresponding to the second set of scanning data is greater than the count threshold.

In the embodiments of the present disclosure, the count rate of coincidence events corresponding to the second set of scanning data can be determined based on the second set of scanning data. On a condition that the count rate of coincidence events corresponding to the second set of scanning data is less than the count rate threshold, the initial single frame duration is further extended. The second set of scanning data corresponding to the extended initial single frame duration are obtained until the obtained count rate of coincidence events corresponding to the second set of scanning data is greater than or equal to the count rate threshold.

In the embodiments of the present disclosure, the count or count rate of coincidence events may also be determined in other ways. In some examples, the count or count rate of coincidence events may be determined based on the theory of the model. Specifically, a mathematical model may be developed to theoretically calculate the count or count rate based on the physical principles and geometry of the PET system, as well as the decay properties of the radiopharmaceutical. For example, parameters such as the decay constant of the radionuclide, the injected dose of the drug, the efficiency of the detector, the attenuation coefficient of the human body, etc., are known, and a formula can be used to calculate the count or count rate of coincidence events that may be detected in an ideal situation.

In some examples, the count or count rate of coincidence events may be determined with reference to previous similar case data, which may include at least one of the same or similar radiopharmaceuticals, scan sites, or patient characteristics. An empirical model or reference range of the count or count rate may be established by analysing the case data. For a new scanned object, pre-estimates are made based on their similar characteristics, such as age, weight, examination site, etc., with reference to count or count rate data from previous cases.

In some examples, the count or count rate of coincidence events can be determined using Monte Carlo simulation, a probabilistic statistics-based method for estimating the count or count rate in PET imaging by simulating processes such as propagation, scattering, and absorption of photons in human tissues and the response of detectors.

In some examples, the count or count rate of coincidence events can be determined based on device performance testing and calibration data. During performance testing and calibration of a PET device, some information is obtained about parameters such as at least one of detector sensitivity, compliance time window, or dead time. Using these known equipment performance parameters, a preliminary estimate of the count or count rate can be made based on information about the radiopharmaceutical. For example, by measuring the count or count rate of the detector with a known radioactive source, a range of possible count or count rates can be extrapolated in the scanned object.

In the embodiments of the present disclosure, when constructing the target single frame image, the second set of scanning data may be processed to reconstruct and obtain the target single frame image.

In some embodiments, the image generated from the second set of scanning data can be fused with images of other modalities (such as CT, MRI, etc.) to help doctors analyze the target part of the scanned object more comprehensively and accurately.

In some examples, taking a first single frame image corresponding to the 1 s˜2 s of the PET dynamic image and the next single frame image of the first single frame image corresponding to the 3 s˜5 s of the PET dynamic image as an example, if the initial single frame duration is extended to obtain the first single frame duration, and then the target single frame image corresponding to the 1 s˜3 s of the PET dynamic image is obtained, so that the next single frame image corresponds to the 4 s˜6 s of the PET dynamic image, i.e., a time period corresponding to the next single frame image is postponed as the target single frame image is determined.

The method provided in the embodiments of the present disclosure includes the following steps. When scanning the target part of the scanned object, the first single frame image corresponding to the initial single frame duration of the PET dynamic image is obtained. On a condition that the count rate of coincidence events corresponding to the first single frame image is less than the count threshold, the second set of scanning data corresponding to the first single frame duration of the PET dynamic image are obtained. The first single frame duration is obtained by extending the initial single frame duration. On a condition that the count of coincidence events corresponding to the second set of scanning data is greater than or equal to the count threshold, the target single frame image of the target part is constructed based on the second set of scanning data, thereby improving the quality of reconstructed target single frame image and improving doctors' work efficiency.

In some embodiments, as shown in FIG. 2, in step S104, constructing the target single frame image of the target part based on the second set of scanning data includes the following steps S1401 to S1403.

In step S1041, an intermediate single frame image of the target part is determined based on the second set of scanning data.

In the embodiments of the present disclosure, the intermediate single frame image is obtained by using image reconstruction technologies such as computed tomography (CT), PET or magnetic resonance imaging (MRI).

In the embodiments of the present disclosure, the SNR is configured to measure a ratio of signal to noise in an image and is usually used to evaluate the quality of an image.

In the embodiments of the present disclosure, a signal area in the intermediate single frame image can first be determined. In medical imaging, the signal area usually refers to an area of human tissue or organ. The signal may be the density, contrast or other characteristics of the tissue. Then a noise level of the image is determined. The noise may be random interference introduced by the imaging device, environmental factors or other sources. Usually, an area containing no signal can be selected in the image to estimate the noise level. After the signal area and the noise level are determined, the SNR can be calculated. The calculation formula of the SNR can be expressed as follows:

$$SNR = Signal / Noise$$

where Signal refers to the intensity of the signal and Noise refers to the intensity of the noise. In medical imaging, specific units (such as Hounsfield units or grayscale) are usually used to describe the intensity of the signal and noise.

In step S1042, a third set of scanning data corresponding to the second single frame duration are obtained when the SNR of the intermediate single frame image is less than the SNR threshold. The second single frame duration is obtained by extending the first single frame duration.

In the embodiments of the present disclosure, the SNR threshold can be configured. According to the specific application scenario and requirements, a SNR threshold can be set to determine whether the image quality meets the quality enhancement requirements. On a condition that the SNR is lower than the SNR threshold, the quality enhancement requirements are met, and adjustment or optimization is required to improve the image quality.

In the embodiments of the present disclosure, on a condition that the SNR is less than the SNR threshold, it can be considered that the image quality is not high, and the first single frame duration needs to be extended to obtain the second single frame duration.

In the embodiments of the present disclosure, the second single frame duration may be 3 seconds.

In the embodiments of the present disclosure, based on the adjusted second single frame duration, more data can be obtained, which helps to increase the SNR and improve the image quality.

In step S1043, the target single frame image is constructed based on the third set of scanning data. A SNR corresponding to the target single frame image is greater than the SNR threshold.

In the embodiments of the present disclosure, the third set of scanning data may be preprocessed, including denoising, filtering, artifact removal and other operations, to reduce noise and other interference. The preprocessed third set of scanning data is then used for image reconstruction, and imaging parameters can be optimized based on the characteristics of the third set of scanning data. Adjustment of the imaging parameters may include adjustment of contrast, brightness, window width, window position and other parameters to ensure optimal performance of image clarity and contrast.

In the embodiments of the present disclosure, the reconstructed image can be analyzed to ensure that the image contains required lesion information of the target part and to evaluate the quality and accuracy of the image. Based on the optimized imaging parameters and analysis results, a target single frame image of the target part can be generated.

According to the method provided in the embodiments of the present disclosure, the PET dynamic image of the target part of the scanned object is obtained. The quality analysis is performed on the PET dynamic image to obtain the quality analysis result. On a condition that the quality analysis result indicates that the first single frame image meets quality enhancement requirements, the first single frame image is obtained, and the first single frame image corresponds to the initial single frame duration. On a condition that the count rate of coincidence events corresponding to the first single frame image is less than the count threshold, the second set of scanning data corresponding to the first single frame duration of the PET dynamic image are obtained. The first single frame duration is obtained by extending the initial single frame duration. The target single frame image of the target part is constructed based on the second set of scanning data. Since a single frame acquisition time is extended, more set of scanning data can be obtained, thereby improving the quality of each reconstructed frame image.

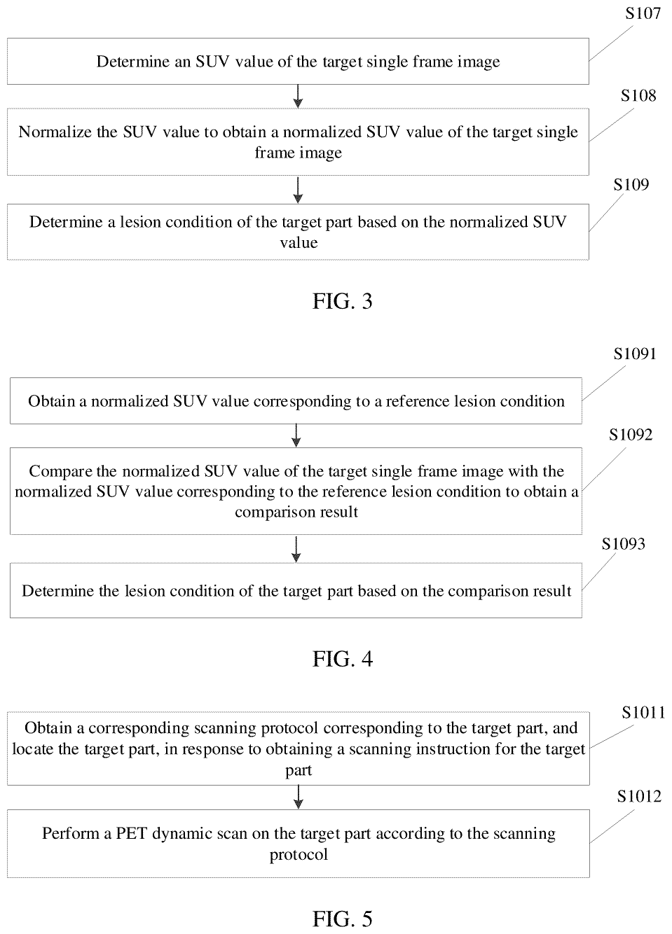

In an embodiment, as shown in FIG. 3, after step S104, the method further includes the following steps S107 to S109.

In step S107, an SUV value of the target single frame image is determined.

In the embodiments of the present disclosure, SUV (Standardized Uptake Value) is an indicator used to assess tumor metabolic activity and is commonly used in quantitative analysis of PET images. In medical imaging, SUV values can help doctors assess tumor activity and treatment response.

In the embodiments of the present disclosure, a region of interest (ROI) can be selected in a target single frame image, which is usually a tumor or other lesion area. An SUV value within the ROI is calculated based on raw data obtained by the PET scan, combined with information such as the patient's weight and the dose of injected radioactive tracer.

In the embodiments of the present disclosure, the region of interest can be determined by using tools in medical image processing software, such as manual delineation or automatic boundary detection algorithms.

In some embodiments, after the SUV value is determined, the SUV value can be determined and calibrated. The SUV value can be calculated repeatedly multiple times to ensure the stability and consistency of the results. This helps to eliminate any deviations due to errors or uncertainties. The parameters used to calculate the SUV value, including a dose of the injected radioactive tracer, a patient weight, a scan time, etc., can be checked to ensure that the parameters are input accurately to avoid deviations in the calculation results.

In step S108, the SUV value is normalized to obtain a normalized SUV value of the target single frame image.

In the embodiments of the present disclosure, the SUV value is normalized and can be considered as a correction base based on a phantom test.

In the embodiments of the present disclosure, the SUV value is affected by many factors, including patient preparation, equipment performance, reconstruction algorithms, etc. Due to the influence of many factors, the SUV value lacks university. Therefore, the calculation of normalized SUV value can eliminate the differences between different patients, different equipment performances, and different reconstruction algorithms, thereby improving the comparability and universality of the SUV value. The normalized SUV value can also be used to evaluate treatment effects and prognosis, and is often used for follow-up and evaluation after PET/CT scans.

The method provided in the embodiments of the present disclosure can achieve cross-device, cross-patient, and cross-time comparability of the SUV value by determining the normalized SUV value, thereby eliminating the impact of differences in measuring instruments in different hospitals, differences in different patients, and differences in different detection times on the test results.

In step S109, a lesion condition of the target part is determined based on the normalized SUV value.

In the embodiments of the present disclosure, when determining the lesion condition of the target part based on the normalized SUV value, it is generally necessary to perform a comprehensive analysis in combination with clinical data and other imaging examination results. In general, parts with higher normalized SUV values may indicate that tumor tissues have more glucose uptake and higher metabolic activity, which may be malignant tumors. Parts with lower normalized SUV values may indicate normal tissues or benign tumors.

In the embodiments of the present disclosure, as shown in FIG. 4, step S109 can be implemented by the following steps.

In step S1091, a normalized SUV value corresponding to a reference lesion condition is obtained.

In the embodiments of the present disclosure, different normalized SUV values correspond to different lesion conditions. The lesion conditions include normal tissues or reference lesions, and the normalized SUV value corresponding to the reference lesion condition may include a normalized SUV value of a normal tissue or a normalized SUV value of a reference lesion.

In step S1092, the normalized SUV value of the target single frame image is compared with the normalized SUV value corresponding to the reference lesion condition to obtain a comparison result.

In the embodiments of the present disclosure, the comparison result may include whether there is a normalized SUV value corresponding to a reference lesion condition that is similar to the normalized SUV value.

In step S1093, the lesion condition of the target part is determined based on the comparison result.

In the embodiments of the present disclosure, if the normalized SUV value is close to that of normal tissue or reference lesion, the lesion condition can be determined.

The method provided in the embodiments of the present disclosure can facilitate multi-modal and multi-scan comparison and verification experiments by normalizing the SUV value.

In an embodiment, as shown in FIG. 5, step S101 includes the following steps S1011 to S1012.

In step S1011, a corresponding scanning protocol corresponding to the target part is obtained, and the target part is located, in response to obtaining a scanning instruction for the target part.

In the embodiments of the present disclosure, a doctor or clinician can directly issue a scanning instruction, so that the electronic device obtains the scanning instruction. In the embodiments of the present disclosure, target parts may include brain, heart, lungs, etc. The scanning protocol includes the specific steps and parameter settings for the scan. Generally, medical staff will perform settings before the scan to obtain the scanning protocol. Parameters may include scanning duration, injection dose, etc.

In the embodiments of the present disclosure, the target part may be determined by using previous image data or through the guidance of other images. For example, previous CT or MRI images can be used to guide the scan position, ensuring that the new scan corresponds to the previous images.

In some embodiments, guide wires or markers may be placed over the target part to facilitate accurate positioning during scanning. These guide wires or markers can be placed by X-ray, ultrasound or other image-guided techniques.

In some embodiments, an image of the user can be obtained and then input into a neural network model to determine the position of the target part, thereby completing the positioning.

For example, if the scanned object is the head, then the brain will be located intelligently as the target organ. If a scan region is the lungs, the aorta can be intelligently located.

In step S1012, a PET dynamic scan is performed on the target part according to the scanning protocol.

In the embodiments of the present disclosure, PET dynamic scan can be automatically performed on the target part according to the scanning protocol.

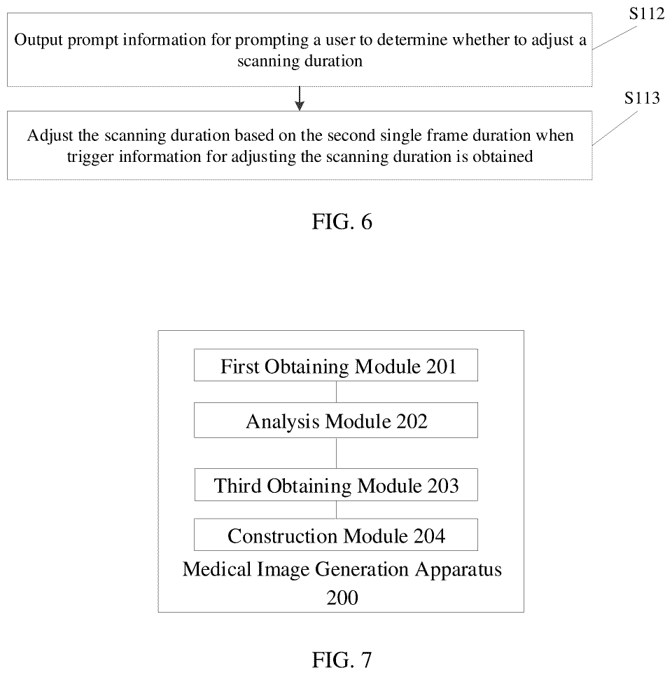

In some embodiments, as shown in FIG. 6, after step S104, the method includes the following steps S112 to S113.

In step S112, prompt information for prompting a user to determine whether to adjust a scanning duration is output.

In the embodiments of the present disclosure, due to the change of the single frame duration, if the number of frames of the obtained image remains unchanged, the scanning duration will also change. Therefore, prompt information may be output to prompt the user whether to adjust the scanning duration.

In the embodiments of the present disclosure, the prompt information can be output through a display device. A selection option can be set in the prompt information, and the selection option is used to select whether to adjust the scanning duration.

In step S113, the scanning duration is adjusted based on the second single frame duration when trigger information for adjusting the scanning duration is obtained.

In the embodiments of the present disclosure, the user can make actual choices based on the actual situation and the operating guide of the device.

In the embodiments of the present disclosure, if the user chooses to adjust the scanning duration, trigger information for adjusting the scanning duration can be obtained.

It should be noted that the medical image generation method provided in the embodiments of the present disclosure can be applied to the long axis view or the short axis view. The long axis view is a view obtained by tomographic imaging in the long axis direction of the organ, and the short axis view refers to a view obtained by scanning the cross section perpendicular to the long axis (such as horizontal plane imaging). The long-axis view generally has one or two beds, covering a plurality parts, and has higher accuracy requirements for the image segmentation algorithm used (for example, the intelligent segmentation algorithm). The short-axis view generally has a plurality beds, and the image quality problems that may occur in different parts of the human body will be different, so targeted detection of the scanning range is required. If the target part of the scan is the head, the region of interest can be determined as the brain, and the brain area can be segmented out by the image segmentation algorithm. The image quality is controlled by the SUV value of the brain area, and the single frame duration and scanning duration are adjusted by the above method. If the target part of the scan is the lung, the region of interest can be determined as the descending aorta, and the descending aorta area can be segmented out by the image segmentation algorithm. The image quality is controlled by the SUV value of the descending aorta area, and the single frame duration and scanning duration are adjusted by the above method. The user can set the ideal reference area for each area separately to obtain the normalized SUV value corresponding to the reference lesion. The image segmentation algorithm can be adapted to the scenes of the long-axis view and the short-axis view respectively through corresponding parameter settings.

It should be noted that in the embodiments of the present disclosure, the adjustment of the single frame duration, scanning duration, etc. can be determined based on artificial intelligence technologies.

In the embodiments of the present disclosure, artificial intelligence technologies can automatically predict a reasonable scanning duration based on the second single frame duration by learning and analyzing a large amount of data.

Based on the aforementioned embodiments, the embodiment of the present disclosure provides a specific example application. During CT-based liver segmentation, the count of coincidence events is determined based on the set of scanning data while performing a scan. On a condition that the count of coincidence events corresponding to the first single frame image is less than the count threshold, an acquisition duration corresponding to the single frame is adjusted, so that more set of scanning data is obtained. By reconstructing the PET image of the liver area with more set of scanning data, the image quality of each frame can be improved. At the same time, due to the change of the single frame acquisition time, the overall scanning duration also needs to be corrected. A count threshold corresponding to different time periods, ages, genders, and weights can be defined to optimize the single frame acquisition time.

The method provided in the embodiments of the present disclosure controls the single-frame duration to achieve image quality control, which can ensure the quality of the image. In addition, by setting the normalized SUV value corresponding to the reference lesion condition for comparison, the standard can be unified, making multi-center image comparison possible.

According to the above-mentioned embodiment, a medical image generation apparatus is provided in the embodiments of the present disclosure, and the modules included in the apparatus and the units included in each module can be implemented by a processor in a computer device. They can also be implemented by a specific logic circuit. In the implementation process, the processor can be a central processing unit (CPU), a microprocessor unit (MPU), a digital signal processor (DSP) or a field programmable gate array (FPGA), etc.

A medical image generation apparatus is provided in the present disclosure. FIG. 7 is a schematic diagram of a configuration of a medical image generation apparatus provided in the embodiments of the present disclosure. As shown in FIG. 7, the medical image generation apparatus 200 includes a first obtaining module 201, an analysis module 202, a third obtaining module 203 and a construction module 204.

The first obtaining module 201 is configured to obtain a PET dynamic image of a target part of a scanned object.

The analysis module 202 is configured to perform quality analysis on the PET dynamic image to determine a first single frame image that meets quality enhancement requirements, the first single frame image corresponding to an initial single frame duration.

The third obtaining module 203 is configured to obtain a second set of scanning data corresponding to a first single frame duration of the PET dynamic image when a count of coincidence events corresponding to the first single frame image is less than a count threshold. The first single frame duration is obtained by extending the initial single frame duration.

The construction module 204 is configured to construct a target single frame image of the target part based on the second set of scanning data. The count rate of coincidence events corresponding to the second set of scanning data is greater than or equal to the count rate threshold.

In some embodiments, the construction module includes a first determination unit, a first obtaining unit, and a construction unit.

The first determination unit is configured to determine an intermediate single frame image of the target part based on the second set of scanning data.

The first obtaining unit is configured to obtain a third set of scanning data corresponding to a second single frame duration when a SNR of the intermediate single frame image is less than a SNR threshold. The second single frame duration is obtained by extending the first single frame duration.

The construction unit is configured to construct the target single frame image based on the third set of scanning data. A SNR corresponding to the target single frame image is greater than the SNR threshold.

In some embodiments, the medical image generation apparatus further includes a first determination module configured to determine the count rate threshold based on body parameters of the scanned object.

In some embodiments, the first determination module includes a fourth obtaining module and a second determination module.

The fourth obtaining module is configured to obtain a pre-stored correspondence and the body parameters. The correspondence includes a correspondence between the body parameters and the count rate threshold.

The second determination module is configured to determine the count rate threshold based on the body parameters and the correspondence.

In some embodiments, the medical image generation apparatus further includes a third determination module, a normalization processing module and a fourth determination module.

The third determination module is configured to determine an SUV value based on the target single frame image.

The normalization processing module is configured to normalize the SUV value to obtain a normalized SUV value of the target single frame image.

The fourth determination module is configured to determine a lesion condition of the target part based on the normalized SUV value.

In some embodiments, the fourth determination module includes a third obtaining unit, a comparison unit, and a fourth determination unit.

The third obtaining unit is configured to obtain a normalized SUV value corresponding to a reference lesion condition.

The comparison unit, is configured to compare the normalized SUV value of the target single frame image with the normalized SUV value corresponding to the reference lesion condition to obtain a comparison result.

The fourth determination unit is configured to determine the lesion condition of the target part based on the comparison result.

In some embodiments, the medical image generation apparatus further includes a fifth obtaining module and a scanning module.

The fifth obtaining module is configured to obtain a corresponding scanning protocol corresponding to the target part, and locate the target part, in response to obtaining a scanning instruction for the target part.

The scanning module is configured to perform a PET dynamic scan on the target part according to the scanning protocol.

In some embodiments, the medical image generation apparatus further includes an output module and a second adjustment module.

The output module is configured to output prompt information for prompting a user to determine whether to adjust a scanning duration.

The second adjustment module is configured to adjust the scanning duration based on the second single frame duration when trigger information for adjusting the scanning duration is obtained.

In some embodiments, the first obtaining module 201 is further configured to determine the target part of the scanned object, perform a PET dynamic scan on the target part to obtain a PET image sequence of the target part, and process the PET image sequence through an image reconstruction algorithm to obtain the PET dynamic image.

In some embodiments, the quality enhancement requirements include one or more of the following: an SUV value, a SNR, a coefficient of variation, or a noise equivalent count rate being less than a set value, or artifacts exceeding a threshold range.

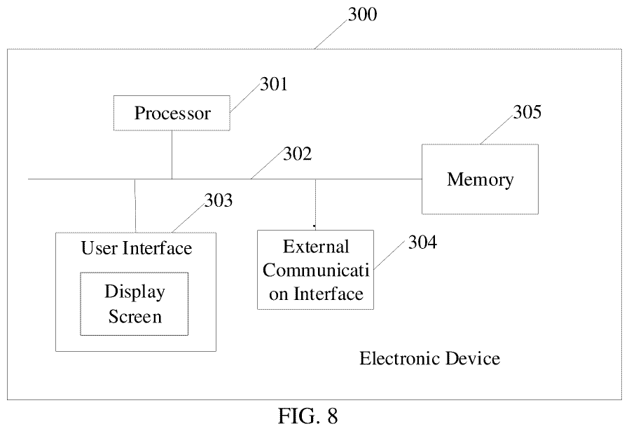

An electronic device is provided in the embodiments of the present disclosure. FIG. 8 is a schematic diagram of a configuration of an electronic device provided in the embodiments of the present disclosure. As shown in FIG. 8, the electronic device 300 includes: a processor 301, at least one communication bus 302, a user interface 303, at least one external communication interface 304, and a memory 305. The communication bus 302 is configured to connect these components for communication. The user interface 303 may include a display screen, and the external communication interface 304 may include a standard wired interface and a wireless interface. The processor 301, when executing the computer program stored in the memory, is configured to perform the medical image generation method provided in any one of the above embodiments.

In the embodiments of the present disclosure, if the above-mentioned medical image generation method is implemented in the form of a software function module and sold or used as an independent product, it can also be stored in a computer-readable storage medium. Based on this understanding, the technical solution of the embodiment of the present disclosure can be essentially or partly reflected in the form of a software product that contributes to the prior art. The computer software product is stored in a storage medium and includes several instructions for a computer device (which can be a personal computer, server, or network device, etc.) to execute all or part of the methods described in each embodiment of the present disclosure. The aforementioned storage medium includes various types of mediums that can store program instructions, for example, U disk, mobile hard disk, read-only memory (ROM, Read Only Memory), disk or optical disk, etc. In this way, the embodiments of the present disclosure are not limited to any specific combination of hardware and software.

In an embodiment, a non-transitory computer-readable storage medium is provided, on which a computer program is stored. A processor, when executing the computer program, performs the medical image generation method in any one of the above-described embodiments.

A computer program product is provided in the embodiments of the present disclosure. The computer program product, when running on an electronic device, causing the electronic device to perform the medical image generation method in any one of the above-described embodiments.

The description of the above electronic device and storage medium embodiments is similar to the description of the above method embodiments, and has similar beneficial effects as the method embodiments. For technical details not disclosed in the computer device and storage medium embodiments of the present disclosure, please refer to the description of the method embodiments of the present disclosure for understanding.

It should be understood that “one embodiment” or “an embodiment” mentioned throughout the specification means that specific features, structures or characteristics related to the embodiment are included in at least one embodiment of the present disclosure. Therefore, “in one embodiment” or “in an embodiment” appearing throughout the specification does not necessarily refer to the same embodiment. In addition, these specific features, structures or characteristics can be combined in one or more embodiments in any suitable manner. It should be understood that in various embodiments of the present disclosure, the numerical order of the above-mentioned processes does not imply a specific sequence of execution. The execution order of each process should be determined by its function and internal logic, and should not constitute any limitation on the implementation process of the embodiments of the present disclosure. The serial number of the above-mentioned embodiments of the present disclosure is only for description and does not represent the advantages and disadvantages of the embodiments.

It should be noted that, in this article, the terms “comprises”, “includes” or any other variations thereof are intended to cover non-exclusive inclusion, so that a process, method, article or apparatus that includes a series of elements includes not only those elements, but also includes other elements not explicitly listed, or also includes elements inherent to such process, method, article or apparatus. In the absence of further restrictions, an element defined by the sentence “including a . . . ” does not exclude the existence of other identical elements in the process, method, article or device including the element.

In the several embodiments provided in the present disclosure, it should be understood that the disclosed devices and methods can be implemented in other ways. The apparatus embodiments described above are only schematic. For example, the division of the units is only a logical function division. There may be other division methods in actual implementation, such as, multiple units or components can be combined, or can be integrated into another system, or some features can be ignored or not executed. In addition, the coupling, direct coupling, or communication connection between the components shown or discussed can be through some interfaces, and the indirect coupling or communication connection of the device or unit can be electrical, mechanical or other forms.

The units described above as separate components may or may not be physically separated, and the components shown as units may or may not be physical units. They may be located in one place or distributed on multiple network units. Some or all of the units may be selected according to actual needs to achieve the purpose of the scheme of this embodiment.

In addition, all functional units in the embodiments of the present disclosure may be integrated into one processing unit, or each unit may be separately configured as a unit, or two or more units may be integrated into one unit. The above-mentioned integrated units may be implemented in the form of hardware or in the form of hardware plus software functional units.

Those of ordinary skill in the art can understand that all or part of the steps in the methods of the above embodiments can be completed by instructing relevant hardware through a computer program. The computer program can be stored in a non-volatile computer-readable storage medium. The computer program, when executed, may implement steps of the method described above in the embodiments. The aforementioned storage media include: mobile storage devices, read-only memories (ROM), magnetic disks or optical disks, and other media that can store program instructions.

Alternatively, if the above-mentioned integrated unit of the present disclosure is implemented in the form of a software function module and sold or used as an independent product, it can also be stored in a computer-readable storage medium. Based on this understanding, the technical solution of the embodiment of the present disclosure can be essentially or partly reflected in the form of a software product, which is stored in a storage medium and includes several instructions for a controller to execute all or part of the methods described in each embodiment of the present disclosure. The aforementioned storage medium includes: various media that can store program instructions, such as mobile storage devices, ROMs, magnetic disks or optical disks.

The technical features of the above embodiments can be randomly combined. To simplify the description, not all possible combinations of the technical features in the above embodiments are described. However, as long as there is no contradiction in the combination of these technical features, all the combinations should be considered to be included within the scope of this specification.

The above-described embodiments only illustrate several embodiments of the present disclosure, and the descriptions of which are relatively specific and detailed, but should not be construed as limiting the scope of the patent disclosure. It should be noted that, for those of ordinary skill in the art, several modifications and improvements can be made without departing from the concept of the present disclosure, and these all fall within the protection scope of the present disclosure. Therefore, the protection scope of the present disclosure should be determined by the appended claims.

What is claimed is:

1. A medical image generation method, comprising:
obtaining a PET dynamic image of a target part of a scanned object;
performing quality analysis on the PET dynamic image to determine a first single frame image that meets quality enhancement requirements, the first single frame image corresponding to an initial single frame duration;
obtaining a second set of scanning data corresponding to a first single frame duration of the PET dynamic image when a count of coincidence events corresponding to the first single frame image is less than a count threshold, wherein the first single frame duration is obtained by extending the initial single frame duration; and
constructing a target single frame image of the target part based on the second set of scanning data, the count of coincidence events corresponding to the second set of scanning data being greater than or equal to the count threshold.

2. The medical image generation method according to claim 1, wherein constructing a target single frame image of the target part based on the second set of scanning data comprising:
determining an intermediate single frame image of the target part based on the second set of scanning data;
obtaining a third set of scanning data corresponding to a second single frame duration when a SNR of the intermediate single frame image is less than a SNR threshold, wherein the second single frame duration is obtained by extending the first single frame duration; and
constructing the target single frame image based on the third set of scanning data, a SNR corresponding to the target single frame image being greater than the SNR threshold.

3. The medical image generation method according to claim 1, further comprising:
determining the count threshold based on body parameters of the scanned object.

4. The medical image generation method according to claim 3, wherein determining the count threshold based on the body parameters of the scanned object comprising:
obtaining a pre-stored correspondence and the body parameters, the correspondence comprising a correspondence between the body parameters and the count threshold; and
determining the count threshold based on the body parameters and the correspondence.

5. The medical image generation method according to claim 1, further comprising:
determining an SUV value of the target single frame image;
normalizing the SUV value to obtain a normalized SUV value of the target single frame image; and
determining a lesion condition of the target part based on the normalized SUV value.

6. The medical image generation method according to claim 5, wherein determining the lesion condition of the target part based on the normalized SUV value comprising:
obtaining a normalized SUV value corresponding to a reference lesion condition;
comparing the normalized SUV value of the target single frame image with the normalized SUV value corresponding to the reference lesion condition to obtain a comparison result; and
determining the lesion condition of the target part based on the comparison result.

7. The medical image generation method according to claim 1, further comprising:
obtaining a corresponding scanning protocol corresponding to the target part, and locating the target part, in response to obtaining a scanning instruction for the target part; and
performing a PET dynamic scan on the target part according to the scanning protocol.

8. The medical image generation method according to claim 2, further comprising:
outputting prompt information for prompting a user to determine whether to adjust a scanning duration; and
adjusting the scanning duration based on the second single frame duration when trigger information for adjusting the scanning duration is obtained.

9. The medical image generation method according to claim 1, wherein obtaining the PET dynamic image of the target part of the scanned object comprising:
determining the target part of the scanned object;
performing a PET dynamic scan on the target part to obtain a PET image sequence of the target part; and
processing the PET image sequence through an image reconstruction algorithm to obtain the PET dynamic image.

10. The medical image generation method according to claim 1, wherein the quality enhancement requirements comprise one or more of the following: an SUV value, a SNR, a coefficient of variation, or a noise equivalent count being less than a set value, or artifacts exceeding a threshold range.

11. An electronic device comprising a memory and a processor, the memory storing a computer program, wherein the processor, when executing the computer program, performs a medical image generation method which comprises:
obtaining a PET dynamic image of a target part of a scanned object;
performing quality analysis on the PET dynamic image to determine a first single frame image that meets quality enhancement requirements, the first single frame image corresponding to an initial single frame duration;
obtaining a second set of scanning data corresponding to a first single frame duration of the PET dynamic image when a count of coincidence events corresponding to the first single frame image is less than a count threshold, wherein the first single frame duration is obtained by extending the initial single frame duration; and
constructing a target single frame image of the target part based on the second set of scanning data, the count of coincidence events corresponding to the second set of scanning data being greater than or equal to the count threshold.

12. The electronic device according to claim 11, wherein the medical image generation method further comprises:
determining an intermediate single frame image of the target part based on the second set of scanning data;
obtaining a third set of scanning data corresponding to a second single frame duration when a SNR of the intermediate single frame image is less than a SNR threshold, wherein the second single frame duration is obtained by extending the first single frame duration; and
constructing the target single frame image based on the third set of scanning data, a SNR corresponding to the target single frame image being greater than the SNR threshold.

13. The electronic device according to claim 11, wherein the medical image generation method further comprises:
determining the count threshold based on body parameters of the scanned object.

14. The electronic device according to claim 13, wherein the medical image generation method further comprises:
obtaining a pre-stored correspondence and the body parameters, the correspondence comprising a correspondence between the body parameters and the count threshold; and
determining the count threshold based on the body parameters and the correspondence.

15. The electronic device according to claim 11, wherein the medical image generation method further comprises:
determining an SUV value of the target single frame image;
normalizing the SUV value to obtain a normalized SUV value of the target single frame image; and
determining a lesion condition of the target part based on the normalized SUV value.

16. The electronic device according to claim 15, wherein the medical image generation method further comprises:
obtaining a normalized SUV value corresponding to a reference lesion condition;
comparing the normalized SUV value of the target single frame image with the normalized SUV value corresponding to the reference lesion condition to obtain a comparison result; and
determining the lesion condition of the target part based on the comparison result.

17. The electronic device according to claim 11, wherein the medical image generation method further comprises:
obtaining a corresponding scanning protocol corresponding to the target part, and locating the target part, in response to obtaining a scanning instruction for the target part; and
performing a PET dynamic scan on the target part according to the scanning protocol.

18. The electronic device according to claim 12, wherein the medical image generation method further comprises:
outputting prompt information for prompting a user to determine whether to adjust a scanning duration; and
adjusting the scanning duration based on the second single frame duration when trigger information for adjusting the scanning duration is obtained.

19. The electronic device according to claim 11, wherein the medical image generation method further comprises:
determining the target part of the scanned object;
performing a PET dynamic scan on the target part to obtain a PET image sequence of the target part; and
processing the PET image sequence through an image reconstruction algorithm to obtain the PET dynamic image.

20. A non-transitory computer-readable storage medium having a computer program stored thereon, wherein the computer program, when executed by a processor, causes the processor to perform a medical image generation method of claim 1.
